(19) United States
(12) Patent Application Publication
(10) Pub. No.: US 2005/0256401 A1
(43) Pub. Date: Nov. 17, 2005

(54) Catheter with variable diameter core spacing and associated actuated device

(75) Inventors: Todd A. Hall, Goshen, KY (US); Raymond Diaz, Weston, FL (US)

Correspondence Address: ROBERT C. KAIN, JR., 750 SOUTHEAST THIRD AVENUE, SUITE 100, FT LAUDERDALE, FL, 333161153, US

(73) Assignee: Scion Cardio-Vascular, Inc.

(21) Appl. No.: 10/846,615

(22) Filed: May 14, 2004

(51) Int. Cl. A61B 5/00

(52) U.S. Cl. 600/434; 600/585

(57) ABSTRACT

The elongated catheter has a movably actuated core wire therein adapted for use with a coaxially actuated medical device. The wire in a tube system has variable core spaces along its axial extent. The catheter includes an elongated catheter tube with a plunge ground core wire movably disposed therein. Otherwise, the core wire may be spiral ground. Additionally, the core wire may have varying outer diameters over its lengths, none larger than the inside diameter of the catheter tube. Further, the core wire may be formed by a plurality of braided wires. Alternatively, or in addition to, the elongated catheter tube may be formed by a plurality of braided wires. In this embodiment, a solid core wire may be movably disposed in the braided catheter tube or the core wire may likewise be braided by a second plurality of braided wires.

The present invention relates to a hollow tube or cannula carrying a core wire having different or variable diameter core spacing (the space between the tube and the core wire) wherein the core wire is movably disposed within the elongated hollow tube or cannula and is adopted for use with a coaxially actuated medical device, and relates to a medical device system. As one example, the hypotube or cannula based medical device with an internal core wire and variable core spacing may be used to activate a distal device during a vascular procedure or a procedure on a carotid artery.

### BACKGROUND OF THE INVENTION

Hypotubes or cannula-based medical devices with internal core wires are used to activate many different, distally located medical devices, herein generally identified as “medical device systems.” The present invention can be adapted for use in a relatively wide variety of “wire in a hollow tube” medical device systems. An exemplary list of medical device systems include: (1) Debris/Foreign Body Retrieval devices—similar to a gooseneck snare and the radially actuated basket (proximally located at or near the end of the medical device system) used to retrieve loose stents, ruptured catheters & angioplasty balloons, etc.; (2) Stone Retrieval devices—similar to stone retrieval baskets used to retrieve gall stones, kidney stones, etc.; (3) Distal (or Embolic) Protection devices—such as SCI-PRO, ANGIOGUARD, GUARDWIRE products which provide protection from debris embolizing during intravascular interventional procedures such as PTCA (Percutaneous Transluminal Coronary Angioplasty) and coronary stenting, carotid stenting, peripheral PTA (Percutaneous Transluminal Angioplasty) and stenting, mechanical thrombectomy, etc.; (4) Biopsy retrieval devices that are manipulated proximally to operate a distal component to retrieve tissue samples; (5) Guidewires used to track & guide devices to percutaneous treatment sites and guidewires with a straightenable J-Tip; (6) Interventional Catheters—such as PTCA, PTA, etc balloon catheters, angiogenisis; (7) Delivery Sheaths—such as those used to deliver Nitinol (self-expanding) stents; (8) Laparoscopic instruments—such as those currently used to remove the gall bladder, appendix, etc.; (9) Temporary IVC Filter catheters such as catheter with a basket at the tip that is temporarily deployed to prevent clots from traveling to the lungs resulting in pulmonary embolisms; and (10) Electrophysiology (EP) devices such as mapping catheters and tip ablation. One example of a coaxially actuated medical device deployed at the distal end of a medical device system is disclosed in U.S. Pat. No. 6,537,296 to Levinson. Other coaxially activated medical devices may be utilized.

As a more specific example, a health professional first introduces a guide wire or catheter (a hollow tube with a movably disposed central core wire) into a vein, artery, or other body cavity of a patient. During catherization of the heart, the guide or catheter is inserted often times into the femoral artery. The hypotube cannula is disposed and moves over the central core wire. The health professional, typically with assistance of various imaging systems, guides the guide wire or catheter through the vascular system of the patient. Occasionally, the guide wire or catheter encounters extremely tortuous pathways in the patient's system.

Sometimes medical devices, mounted on a distal end portion of the medical device system, are coaxially actuated based upon longitudinal movement of the core wire with respect to the cannula. These medical device systems are sometimes difficult to deploy and the health professional may encounter resistance in the longitudinal movement of the core wire with respect to the hollow tube (cannula). This resistance adversely effects the coaxially deployment or retraction of the medical instrument at the distal end of the catheter system. The problem is due to the number of twists and turns of the medical device system (the elongated hollow tube and the core wire) deployed in a tortuous body system, such as the vascular system. The sum of the friction, caused by the core wire contacting the inside surface of the elongated cannula, adversely effects the deployment and retraction of coaxially actuated medical device at the distal end of the system. These coaxially actuated medical devices, such as a basket, filter, balloon, stent or other coaxial medical elements, are opened and closed based upon relatively small axial movement of the tube with respect to the central core wire. Since the medical device system twists and turns in the patient and since the total frictional force is the sum of all areas of contact between the hollow tube and the central core wire, which contact areas increase dependent upon the degree of curvature of each twist or turn and number of tortuous incidents along the length of the system, this increase in the total friction of the system adversely effects the deployment and retraction of the basket or other medical devices at the distal end of the system. Essentially, the total friction is the sum of all areas of contact between the outer diameter of the core wire and the inner diameter of the cannula or hollow tube. This is the interior core space. The greater the length of the tortuous pathway and/or the greater the number of contact points, the greater the sum of friction along the length of the system and the greater amount of force required to overcome that friction in order to deploy or retract axially actuated medical devices at the distal end of the system (to move one wire relative to the other).

A a lubricious coating such as Teflon or PTFE or silicon, on the central core wire may reduce this friction.

### OBJECTS OF THE INVENTION

It is an object of the present invention to provide a core wire in a hollow tube with variable core space dimensions (the space between the central wire and the tube) thereby reducing the friction points between the outer dimension of the core wire and the inner dimension of the elongated hollow tube (hypo-tube).

It is a further object of the present invention to provide a plunge ground core wire.

It is an additional object of the present invention to provide a core wire which is plunged ground at periodic intervals.

It is a further object of the present invention to provide a core wire that is braided thereby providing a multiplicity of core wire outer diameters.

It is a further object of the present invention to provide an elongated tube or cannula formed of a plurality of braided wires.

It is an additional object of the present invention to provide a medical device system with a hollow tube formed with a plurality of braided wires and/or a core wire formed of a different plurality of braided wires.

It is a further object of the present invention to provide an elongated core wire having various outer diameters over its length, none larger than the inside diameter of the hollow tube.

It is another object of the present invention to provide a coaxial actuated medical device with the variable dimension core wire.

### SUMMARY OF THE INVENTION

The elongated medical device system with a hollow tube has a movably actuated core wire therein adapted for use with a coaxially actuated medical device. The space between the central wire and the hollow tube is variable such that at different longitudinal positions, the radial space is different. One medical device system includes an elongated hollow tube with a plunge ground core wire movably disposed therein. Otherwise, the core wire may be spiral ground. Additionally, the core wire may have varying outer diameters over its lengths, none larger than the inside diameter of the tube. Further, the core wire may be formed by a plurality of braided wires. Alternatively, or in addition to, the elongated tube may be formed by a plurality of braided wires. In this embodiment, a solid core wire may be movably disposed in the braided tube or the core wire may likewise be braided by a second plurality of braided wires.

### BRIEF DESCRIPTION OF THE DRAWINGS

Further objects and advantages of the present invention can be found in the detailed description of the preferred embodiments when taken in conjunction with the accompanying drawings in which:

FIG. 1 diagrammatically illustrates the core wire having various diameters, and particularly a plunge ground core wire;

FIG. 2A diagrammatically illustrates a slope transition or a tapered transition for the variable diameter core wire;

FIG. 2B diagrammatically illustrates a spiral ground core wire;

FIG. 3 diagrammatically illustrates one example of a coaxially actuated medical device mounted at the distal end of a medical device system;

FIG. 4 diagrammatically illustrates the core wire with variable diameters and a “floppy tip” mounted at its distal end;

FIG. 5 diagrammatically illustrates a cross-sectional view of the variable diameter core wire and an exaggerated lubricious coating layer on the core wire;

FIG. 6A diagrammatically illustrates a braided cannula or tube and FIG. 6B is a cross-section thereof;

FIG. 7A diagrammatically illustrates a braided core wire (with and without a solid core center wire) and FIG. 7B is a cross-section thereof;

FIG. 8 diagrammatically illustrates a cross-sectional view of a solid core wire in an hypo tube or cannula in a tortuous pathway generating high surface friction accumulated over the illustrated length; and

FIG. 9 diagrammatically illustrates a cross-section of a solid core wire in a braided cannula in a tortuous pathway wherein the total wire-to-tube surface, subject to friction, is reduced due to reduction in wire to tube contact between the outer diameter of the core wire and the inner diameter of the cannula (tube)(the same reduction of contact is obtained with the variable diameter core wire of FIG. 1 in the catheter tube of FIG. 8).

### DETAILED DESCRIPTION OF THE PREFERRED EMBODIMENTS

The present invention relates to a wire within a tube medical device system having variable core spaces therein. A variable diameter core wire is deployed in an elongated tube. In another embodiment, a braided tube is utilized (with or without the variable diameter core wire). The wire-in-a-tube configuration reduces the total surface contact in the lumen and hence reduces the total frictional force prohibiting movement of the core wire with respect to the tube. An associated medical device employing the reduced friction core wire and tube is also disclosed.

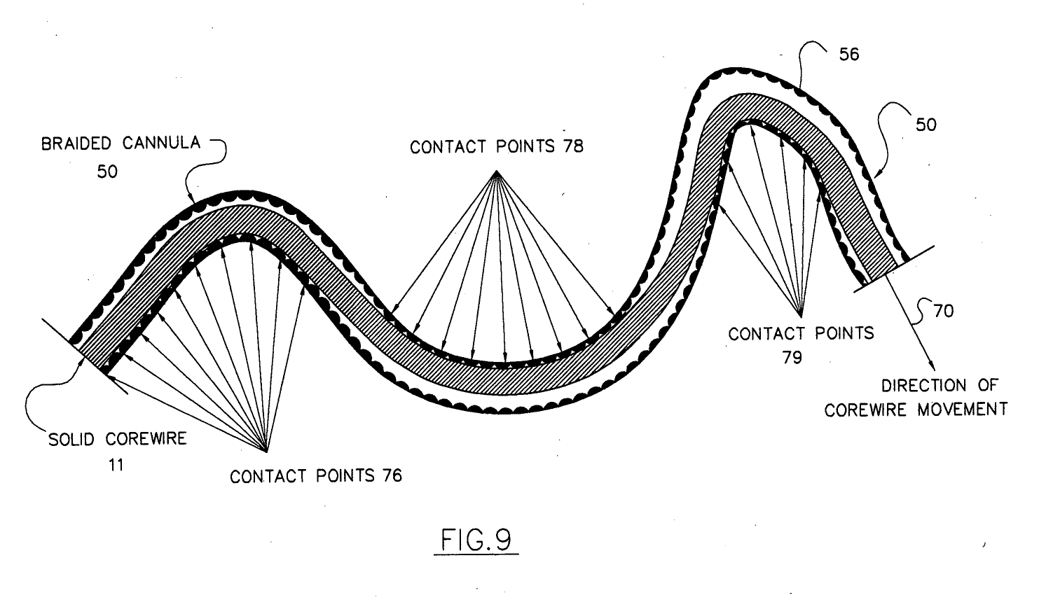

FIG. 1 diagrammatically illustrates core wire 10 having variable or various outer diameters along its length. Core wire 10, in one embodiment, is a solid wire which is plunge ground at regions 12, 14 and at other regions, either periodically or as indicated by the manufacturer, along the axial length of wire 10. In one embodiment, the axial length of wire 10 is 197 cm. The outer diameter of core wire 10, that is, diameter 16, is 0.0191 cm (0.0075 inches). The plunge ground regions 12, 14 and others (not shown) reduce the outer diameter of wire 10 to 0.0013 cm (0.005 inches). The core space at regions 12, 14 is radially larger than the adjacent core spaces therefore there is less contact or less radial force caused by the wire rubbing against the interior of the tube. The purpose of the present invention is to reduce total high friction generating contact areas along the length of the wire-in-a-tube. See FIG. 9. In one embodiment, the axial length 18 of reduced diameter regions 12, 14 is 3-4 mm. Interval spacing 20 between plunge ground regions or reduced diameter regions 12, 14 is, in one embodiment, 1-2 cm. In one embodiment, the reduced diameter regions 12, 14 and the associated interval 20 repeat for 40 cm along the length of core wire 10. In one embodiment, core wire 10 is previously coated with a lubricious coating such as Teflon (PTFE) or silicon. Thereafter, wire 10 is plunge ground to achieve reduced diameter regions 12, 14 (among others). Core wire 10 has a tapered region at distal segment 18 and a tip region 20. Tip region 20 has an outside diameter 21 of 0.0102 cm (0.004 inches).

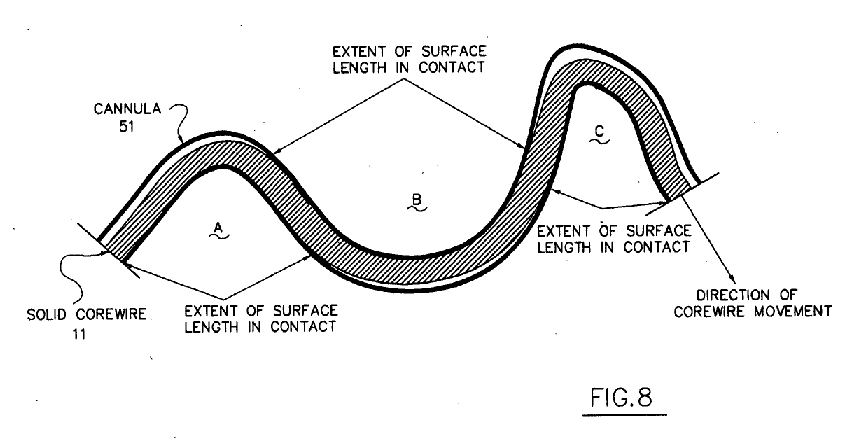

In another embodiment, the core wire may be built up with various coatings of low friction materials such as Teflon (PTFE). Small discrete lengths of shrink type PTFE or similar low friction material could be applied to a smaller mandrel or core wire. The areas of reduced diameter 12, 14 decrease the points of drag and contact between core wire 10 and the elongated hollow tube. FIG. 8 shows lengthy contact regions and FIG. 9 shows reduced contact regions. Variable diameter core wire 10 may be deployed in the hollow tube of FIG. 8 or FIG. 9.

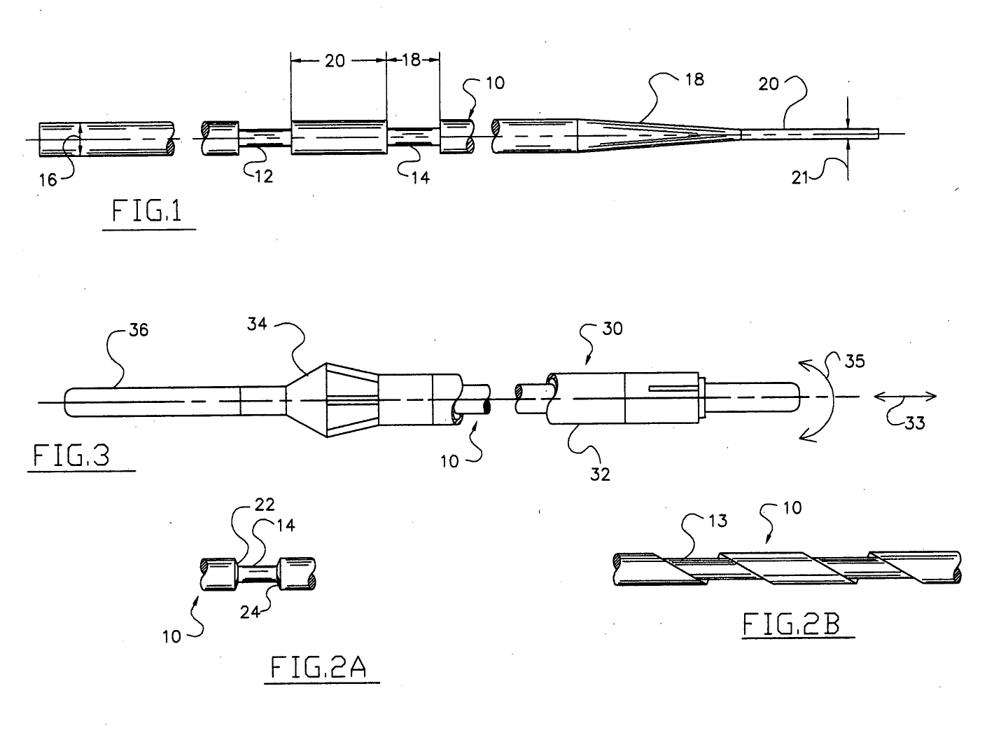

FIG. 2A diagrammatically shows that the transition between larger diameter regions and reduced diameter regions 14, that is, transitions 22, 24 may be tapered or sloped.

FIG. 2B diagrammatically illustrates that core wire 10 may be spiral ground such that reduced diameter region 13 is formed in a spiral manner along a predetermined length of core wire 10.

FIG. 3 diagrammatically illustrates a medical device system 30 utilizing a core wire 10 having variable outer diameters. Core wire 10 is deployed within an elongated hollow tube or cannula 32. A coaxially actuated medical device 34 is mounted on or in connection with tube 32 at or near the distal end 36 of the medical device system 30. FIG. 3 shows, as an example, basket 34 in its opened or fully deployed position. If the physician moves tube 32 relative to core wire 10, medical device or basket 36 closes or opens. Therefore, the medical device 34 is coaxially actuated (radially deployed) to an operative state based upon longitudinal movement of the wire with respect to the tube shown by double headed arrow 33. To collapse coaxial device 34, the wire moves axially (longitudinally) with respect to the tube 32. Further, the health professional may rotate the entire medical device system as shown by curved arrows 35.

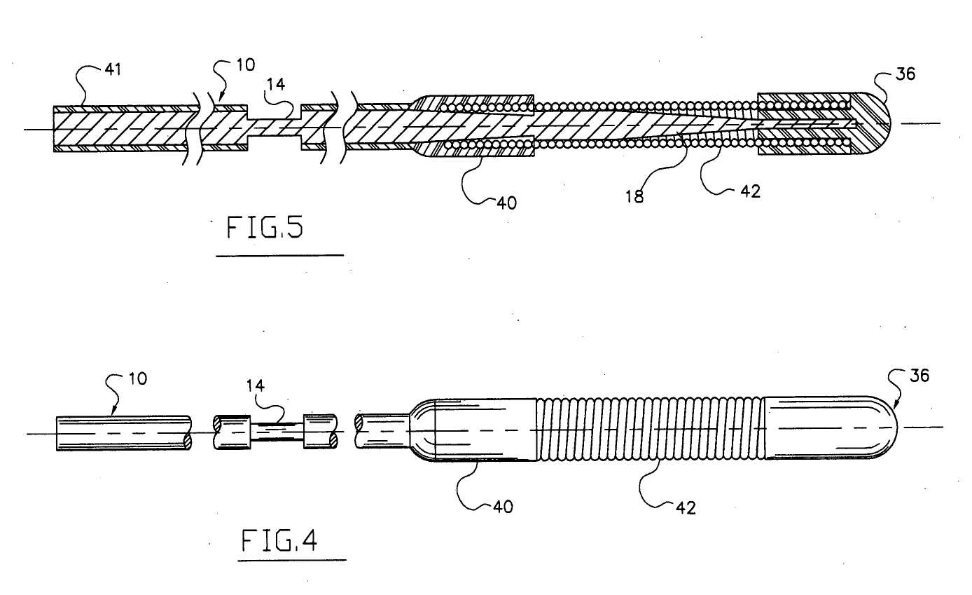

FIG. 4 diagrammatically illustrates core wire 10 having a tip 40 (sometimes called a “floppy tip”) mounted on its distal end 36. Similar numerals designate similar items throughout the figures.

FIG. 5 diagrammatically illustrates a cross-section of core wire 10 and tip 40 and greatly exaggerates the very thin lubricious coating 41 on core wire 10. In one embodiment, PTFE coating 41 is 0.00127 cm (0.0005 inches) thick. FIGS. 4 and 5 are described concurrently herein. Floppy tip 40 includes a spiral body tube 42 soldered at proximal end 44 and soldered at distal end 46. There is a small gap between the interior diameter of spiral tube body 42 and tapered region 18 of core wire 10.

FIG. 5 also shows that reduced diameter region 14 (and other reduced diameter regions not shown in the figures) does not have the lubricious coating thereon. Typically, the core wire is delivered to the device manufacturer with the lubricious coating on the wire and thereafter reduced diameter sections 12, 14 or spiral ground section 13 is created with a plunge grinder or spiral grinder.

In a preferred embodiment, spiral tube tip body 42 is radio opaque and, in a working embodiment, is platinum-iridium, such that the health professional can locate the tip with the imaging system. The floppy tip 42 is not typically coated with lubricious coating.

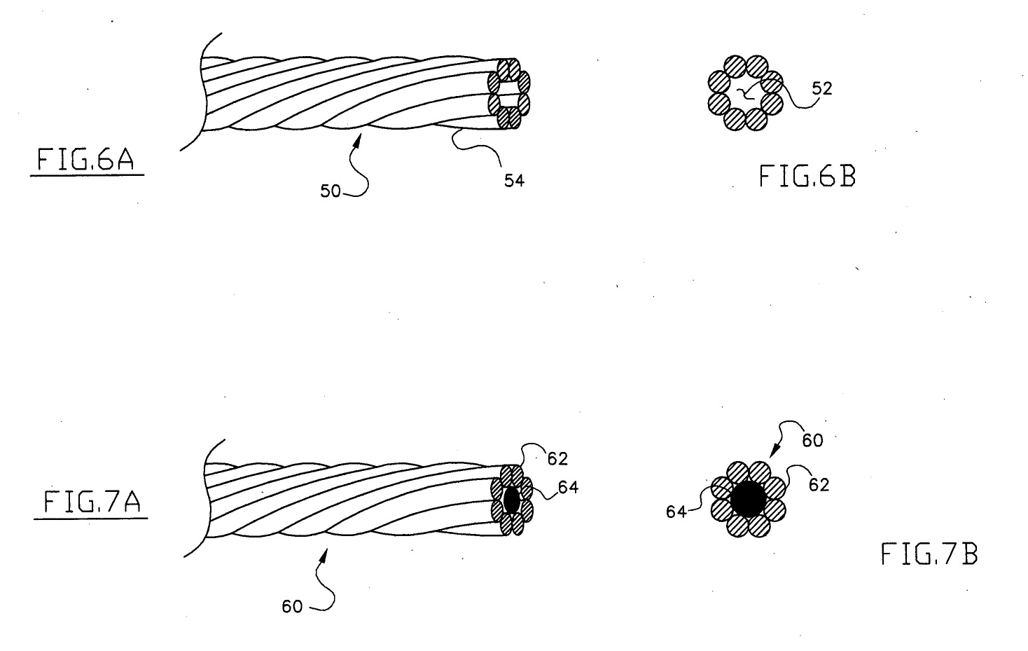

Another technique to reduce the friction points between the core wire (or central wire) and the tube is to form tube 50 with a plurality of braided wires. FIGS. 6A, 6B show a braided wire tube with a passage way 52 within which may be disposed a constant diameter core wire or a core wire having a variable diameter (as described in FIGS. 1, 2B, 4 and 5 above). Braided tube 50 is formed by a plurality of braided wires 54. Braided wires 54 may or may not be coated with the lubricious coating such as Teflon, PTFE, silicon or otherwise. Alternatively, the braided cannula 50 may have its outer diameter coated after it is braided. Preferably, the individual wires 54 are coated prior to braiding such that the lubricious coating will exist on both the inside diameter and the outside diameter of cannula 50. As shown in FIG. 9, the outer surface 56 of braided cannula 50 may be centerless ground in order to provide a flat surface on the outside diameter of the cannula or tube. The valleys in the braids provide the reduced core space between the wire and the hollow tube.

FIGS. 7A, 7B diagrammatically illustrate a braided core wire 60 formed of a plurality of wires 62 braided together. FIG. 7B shows a cross-sectional view of braided core wire 60. Center wire 64 may be a single wire running the axial length of braided core 60 or may be one of the plurality of braided wire 62. Individual wires 62, 64 may be coated with a lubricious coating or the entire braided core wire 60 may have its outside diameter coated with such coating after braiding.

Additionally, the braided core wire 60 may be utilized in conjunction with the braided catheter tube or cannula 50. Implementing the braided tube or cannula in FIG. 6A with a solid core wire reduces the total surface subject to contact during the tortuous pathways encountered by the medical device system. Likewise, a braided core wire as shown in FIG. 7A operating in a common, smooth bore tube (FIG. 3) reduces the total surface area subject to friction. The combination of a braided core wire 60 in a braided tube 50 may also reduce total friction.

FIG. 8 diagrammatically shows simple cannula 51 in a moderately tortuous path. A solid core wire 11 is deployed in cannula 51. As shown, regions A, B and C are subject to high contact which increases the frictional force and impedes longitudinal movement of core wire in direction 70 relative to cannula 51.

FIG. 9 shows braided cannula 50 with solid core wire 11 with a multiplicity of contact points in potential contact regions 76, 78 and 79. The reduction in the surface area contact between solid core wire 11 and braided cannula 50 (variable core space) reduces the total accumulated friction over this tortuous path. The same effect (reduction in contact area and similar reduction in total friction) is achieved utilizing variable diameter core wire 10 shown in FIGS. 1, 2A and 4 in a simple cannulas, (FIG. 8) or a braided cannula 50 (FIG. 6A).

The claims appended hereto are meant to cover modifications and changes within the scope and spirit of the present invention. What is claimed is:

1. An elongated catheter with a movably actuated core wire therein adapted for use with a coaxially actuated medical device comprising an elongated tube with a plunge ground core wire movably disposed therein.

2. A catheter as claimed in claim 1 wherein said core wire is plunge ground at intervals therealong.

3. A catheter as claimed in claim 1 wherein said core wire is plunge ground at periodic intervals therealong.

4. A catheter as claimed in claim 1 wherein said core wire defines plunge ground regions and each plunge ground region has either a straight edge transition, a sloped transition or a tapered transition.

5. A catheter as claimed in claim 1 wherein said core wire is solid with plunge ground regions along portions of its length.

6. An elongated catheter with a movably actuated core wire therein adapted for use with a coaxially actuated medical device comprising an elongated tube with a spiral ground core wire movably disposed therein.

7. A catheter as claimed in claim 6 wherein said core wire is spiral ground at periodic intervals therealong.

8. A catheter as claimed in claim 6 wherein said core wire is solid with spiral ground regions along portions of its length.

9. An elongated catheter with a movably actuated core wire therein adapted for use with a coaxially actuated medical device comprising an elongated tube having an inside diameter, said tube operable with an elongated core wire having varying outer diameters over its length, none larger than said inside diameter of said tube, said core wire being movably disposed therein.

10. A catheter as claimed in claim 9 wherein said varying outer diameters of said core wire include at least a first smaller diameter and a second larger diameter, said larger diameter being less than said inside diameter of said tube, said smaller diameter defined at intervals along said core wire.

11. A catheter as claimed in claim 10 wherein small diameters are defined on said core wire at periodic intervals therealong.

12. A catheter as claimed in claim 10 wherein transition regions from said large diameter to said small diameter are either a straight edge transition, a sloped transition or a tapered transition.

13. A catheter as claimed in claim 10 wherein said smaller diameter regions are defined along portions of the length of said core wire.

14. A catheter as claimed in claim 10 wherein said larger diameters are built-up regions on said core wire.

15. An elongated catheter with a movably actuated core wire therein adapted for use with a coaxially actuated medical device comprising an elongated tube formed by a plurality of braided wires and a solid core wire movably disposed therein.

16. A catheter as claimed in claim 15 wherein each said braided wire forming said tube has a lubricous coating thereon.

17. A catheter as claimed in claim 15 wherein said tube has an outer diameter which is centerless ground to form at least one flat surface thereon.

18. An elongated catheter with a movably actuated core wire therein adapted for use with a coaxially actuated medical device comprising an elongated tube and a core wire formed by a plurality of braided wires, said core wire movably disposed in said tube.

19. A catheter as claimed in claim 18 wherein each said braided wire forming said core wire has a lubricous coating thereon.

20. A catheter as claimed in claim 18 wherein said core wire includes a center wire about which is braided said plurality of braided wires.

21. A catheter as claimed in claim 20 wherein each said braided wire forming said core wire has a lubricous coating thereon.

22. A catheter as claimed in claim 18 wherein said core wire formed by a plurality of braided wires has a lubricous coating thereon.

23. A catheter as claimed in claim 18 wherein said catheter tube is formed by a plurality of braided wires.

24. A catheter as claimed in claim 23 wherein each said braided wire forming said tube has a lubricous coating thereon.

25. A catheter as claimed in claim 24 wherein said tube has an outer diameter which is centerless ground to form at least one flat surface thereon.

26. A medical device system comprising: an elongated hollow tube carrying at its distal end a coaxially actuated medical device, said tube having a plunge ground core wire movably disposed therein, and said coaxially actuated medical device operated by said tube and core wire.

27. A medical device system as claimed in claim 26 wherein said core wire is plunge ground at intervals therealong.

28. A medical device system as claimed in claim 26 wherein said core wire defines plunge ground regions and each plunge ground region has either a straight edge transition, a sloped transition or a tapered transition.

29. A medical device system comprising: an elongated tube carrying at its distal end a coaxially actuated medical device, said tube having a spiral ground core wire movably disposed therein, and said coaxially actuated medical device operated by said tube and core wire.

30. A medical device system as claimed in claim 29 wherein said core wire is spiral ground at periodic intervals therealong.

31. A medical device system as claimed in claim 29 wherein said core wire is solid with spiral ground regions along portions of its length.

32. A medical device system comprising: an elongated tube carrying at its distal end a coaxially actuated medical device, said tube having an inside diameter, an elongated core wire moveably disposed in said tube, said core wire having varying outer diameters over its length, none larger than said inside diameter of said tube, and said coaxially actuated medical device operated by said tube and core wire.

33. A medical device system as claimed in claim 32 wherein said varying outer diameters of said core wire include at least a first smaller diameter and a second larger diameter, said larger diameter being less than said inside diameter of said tube, said smaller diameter defined at intervals along said core wire.

34. A medical device system as claimed in claim 33 wherein small diameters are defined on said core wire at periodic intervals therealong.

35. A medical device system as claimed in claim 33 wherein transition regions from said large diameter to said small diameter are either a straight edge transition, a sloped transition or a tapered transition.

36. A medical device system as claimed in claim 32 wherein said smaller diameter regions are defined along portions of the length of said core wire.

37. A medical device system as claimed in claim 33 wherein said larger diameters are built-up regions on said core wire.

38. A medical device system comprising: an elongated tube carrying at its distal end a coaxially actuated medical device, said tube formed by a plurality of braided wires and a solid core wire movably disposed therein, and said coaxially actuated medical device operated by said tube and core wire.

39. A medical device system as claimed in claim 38 wherein each said braided wire forming said tube has a lubricous coating thereon.

40. A medical device system as claimed in claim 38 wherein said catheter tube has an outer diameter which is centerless ground to form at least one flat surface thereon.

41. A medical device system comprising: an elongated tube carrying at its distal end a coaxially actuated medical device, a core wire formed by a plurality of braided wires, said core wire movably disposed in said tube, and said coaxially actuated medical device operated by said tube and core wire.

42. A medical device system as claimed in claim 41 wherein each said braided wire forming said core wire has a lubricous coating thereon.

43. A medical device system as claimed in claim 41 wherein said core wire includes a center wire about which is braided said plurality of braided wires.

44. A medical device system as claimed in claim 43 wherein each said braided wire forming said core wire has a lubricous coating thereon.

45. A medical device system as claimed in claim 41 wherein said core wire formed by a plurality of braided wires has a lubricous coating thereon.

46. A medical device system as claimed in claim 41 wherein said catheter tube is formed by a plurality of braided wires.

47. A medical device system as claimed in claim 46 wherein each said braided wire forming said catheter tube has a lubricous coating thereon.

48. A medical device system as claimed in claim 47 wherein said catheter tube has an outer diameter which is centerless ground to form at least one flat surface thereon.

49. A medical device system with a distally mounted, coaxially actuated medical device thereat comprising an elongated hollow tube with a central core wire with a plunge ground core wire movably disposed therein.

50. A cannula with a movably actuated core wire therein adapted for use with a coaxially actuated medical device comprising an elongated tube with a plunge ground core wire movably disposed therein.

51. A cannula with a movably actuated core wire as claimed in claim 50 wherein said core wire is plunge ground at intervals therealong.

52. A cannula with a movably actuated core wire as claimed in claim 50 wherein said core wire is plunge ground at periodic intervals therealong.

53. A cannula with a movably actuated core wire as claimed in claim 50 wherein said core wire defines plunge ground regions and each plunge ground region has either a straight edge transition, a sloped transition or a tapered transition.

54. A cannula with a movably actuated core wire as claimed in claim 50 wherein said core wire is solid with plunge ground regions along portions of its length.

55. A cannula with a movably actuated core wire therein adapted for use with a coaxially actuated medical device comprising an elongated tube with a spiral ground core wire movably disposed therein.

56. A cannula with a movably actuated core wire as claimed in claim 55 wherein said core wire is spiral ground at periodic intervals therealong.

57. A cannula with a movably actuated core wire as claimed in claim 55 wherein said core wire is solid with spiral ground regions along portions of its length.

58. A cannula with a movably actuated core wire therein adapted for use with a coaxially actuated medical device comprising an elongated tube having an inside diameter, said tube operable with an elongated core wire having varying outer diameters over its length, none larger than said inside diameter of said tube, said core wire being movably disposed therein.

59. A cannula with a movably actuated core wire as claimed in claim 58 wherein said varying outer diameters of said core wire include at least a first smaller diameter and a second larger diameter, said larger diameter being less than said inside diameter of said tube, said smaller diameter defined at intervals along said core wire.

60. A cannula with a movably actuated core wire as claimed in claim 59 wherein small diameters are defined on said core wire at periodic intervals therealong.

61. A cannula with a movably actuated core wire as claimed in claim 59 wherein transition regions from said large diameter to said small diameter are either a straight edge transition, a sloped transition or a tapered transition.

62. A cannula with a movably actuated core wire as claimed in claim 59 wherein said smaller diameter regions are defined along portions of the length of said core wire.

63. A cannula with a movably actuated core wire as claimed in claim 59 wherein said larger diameters are built-up regions on said core wire.

64. A cannula with a movably actuated core wire therein adapted for use with a coaxially actuated medical device comprising an elongated tube formed by a plurality of braided wires and a solid core wire movably disposed therein.

65. A cannula with a movably actuated core wire as claimed in claim 64 wherein each said braided wire forming said tube has a lubricous coating thereon.

66. A cannula with a movably actuated core wire as claimed in claim 64 wherein said tube has an outer diameter which is centerless ground to form at least one flat surface thereon.

67. A cannula with a movably actuated core wire therein adapted for use with a coaxially actuated medical device comprising an elongated tube and a core wire formed by a plurality of braided wires, said core wire movably disposed in said tube.

68. A cannula with a movably actuated core wire as claimed in claim 67 wherein each said braided wire forming said core wire has a lubricous coating thereon.

69. A cannula with a movably actuated core wire as claimed in claim 67 wherein said core wire includes a center wire about which is braided said plurality of braided wires.

70. A cannula with a movably actuated core wire as claimed in claim 69 wherein each said braided wire forming said core wire has a lubricous coating thereon.

71. A cannula with a movably actuated core wire as claimed in claim 67 wherein said core wire formed by a plurality of braided wires has a lubricous coating thereon.

72. A cannula with a movably actuated core wire as claimed in claim 67 wherein said tube is formed by a plurality of braided wires.

73. A cannula with a movably actuated core wire as claimed in claim 72 wherein each said braided wire forming said tube has a lubricous coating thereon.

74. A cannula with a movably actuated core wire as claimed in claim 73 wherein said tube has an outer diameter which is centerless ground to form at least one flat surface thereon.
